(19) United States
(12) Patent Application Publication
(10) Pub. No.: US 2017/0049698 A1
(43) Pub. Date: Feb. 23, 2017

(54) NATURAL SUPPLEMENT

(71) Applicant: Elina Fuhrman, Studio City, CA (US)

(72) Inventor: Elina Fuhrman, Studio City, CA (US)

(21) Appl. No.: 15/239,699

(22) Filed: Aug. 17, 2016

Related U.S. Application Data
(60) Provisional application No. 62/206,163, filed on Aug. 17, 2015.

(51) Int. Cl. A61K 9/00; A61K 36/48; A61K 36/23; A61K 36/906; A61K 36/235; A61K 36/31; A61K 36/81; A61K 36/185; A61K 36/53; A61K 36/9066; A61K 36/07; A61K 36/70; A23L 23/00; A23L 19/00; A61K 36/42

(52) U.S. Cl. CPC A61K 9/0087; A61K 36/42; A61K 36/48; A61K 36/23; A61K 36/906; A61K 36/235; A61K 36/31; A61K 36/81; A61K 36/185; A61K 36/53; A61K 36/9066; A61K 36/07; A61K 36/70; A23L 23/00; A23L 19/00; A23V 2002/00

(57) ABSTRACT

A supplement including a plurality of ingredients selected from one or more of vegetables, roots, herbs, spices, legumes in combination with a water broth, the plurality of ingredients selected for a nutritional benefit. A method including combining a plurality of ingredients selected from one or more of vegetables, roots, herbs, spices, legumes with a water broth, the plurality of ingredients selected for a nutritional benefit; and simmering the combination for a time period sufficient to release nutrients from the plurality of ingredients.

### CROSS-REFERENCE TO RELATED APPLICATION

The application claims the benefit of the earlier filing date of co-pending U.S. Provisional Patent Application No. 62/206,163, filed Aug. 17, 2015 and incorporated herein by reference.

### FIELD

Natural Supplements.

### BACKGROUND

Soup is a primarily liquid food that is made by combining ingredients such as meat and vegetables with stock, juice, water, or another liquid. Hot soups are generally characterized by boiling solid ingredients in liquids in a pot until the flavors are extracted, forming a broth. One common practice is to use sub-standard ingredients in soups and retain the good or first rate ingredients for salads, side dishes and entrees. According to this practice, soups are often cooked in vegetable or chicken/meat stock to enhance the flavor of one or more sub-standard ingredients.

### DETAILED DESCRIPTION

Soups offering therapeutic benefits are described. A therapeutic benefit as used herein is the provision of one or more nutrients derived from natural food sources intended to balance a diet or regulate or increase or boost a particular system in the body (e.g., immune system, digestive system, circulatory system). In one embodiment, a soup includes ingredients (e.g., vegetables, roots, herbs, spices) selected of first rate quality and combined according to provide desired therapeutic benefits. The ingredients are combined in water and simmered for a period of time sufficient to release the vitamins, minerals and nutrients associated with the ingredients.

1. Water vs. Broth

As noted above, soups are generally cooked in vegetable or chicken/meat stock to enhance the flavors of sub-standard ingredients. In one embodiment, a soup includes good or first rate ingredients simmered in water, preferably purified or filtered water.

Simmering in purified or filtered water allows the best, organic, seasonal and local veggies, roots, spices and herbs to release their flavors and medicinal powers through simmering.

2. Simmering vs. Boiling

Conventional soup recipes require ingredients (e.g., vegetables, roots, spices, herbs) to be placed in broth and boiled. Such boiling kills or otherwise destroys nutrients and medicinal power of the nutrient-dense herbs, veggies, roots, mushrooms, legumes and spices. According to one embodiment, a soup is simmered rather than boiled. A soup with a therapeutic benefit may be simmered in water for hours, sometimes up to 10 hours for larger pots to allow all release of nutrients. Also, by simmering, the soup enhances the absorption of the minerals and vitamins present in the soup in a subject after consumption. Many vegetables only become healthful once simmered properly, due to boosting the medicinal potency through the process.

3. Soaking/Sprouting/Lactic Acid Fermenting

Soaking, sprouting and fermenting of specific vegetables, roots, legumes, spices and herbs enhances the release of amino acids and proper absorption of minerals and vitamins in our bodies. Fermenting changes foods into probiotics and studies have found that the metabolic process of fermentation (converting carbohydrates like sugars into organic lactic acids) creates good bacteria for a subject's microbiome and has a tendency to heal the gut. Soups with probiotic-rich ingredients suppress the growth and even invasion of potentially pathogenic bacteria by producing antimicrobial substances called bacteriocins. As these probiotics bacteria metabolize their sources of fuel from the diet, they liberate various nutrients contained uptaken the foods, making absorption of these nutrients easier. Representatively, the metabolism of probiotic bacteria increases the availability of vitamins A, C and K as well as vitamins from the B-complex group.

Representative of a fermentation process is the lacto-fermenting of Asian pear that goes into a healing broth. Four whole pears are placed in a large jar or pot. Himalayan pink salt, fresh lemon juice, ginger, fennel seeds and dill seeds are added. Room temperature filtered water is added to cover the pears completely but leaving one inch below the top of the jar or pot. The jar or pot is covered with cheesecloth and left standing away from sunlight for up to four days. Then the jar or pot is moved into the fridge to stop fermenting.

Representative of a soaking process is the soaking of shiitake mushrooms for at least an hour before incorporating into a soup, because studies have shown that dried shiitake mushrooms have more anti-cancer benefits than fresh mushrooms. Shiitake mushrooms have been found to inhibit increases in tumor volume, lower blood pressure and reduce cholesterol. They possess powerful antioxidant properties that help the body fight the damage of free radicals. Shiitake mushrooms are used as an anti-cancer drug in Japan. In another embodiment, lentils are soaked overnight before incorporating such lentils in a soup.

In terms of sprouting, lentils, fenugreek, quinoa and radish are sprouted for incorporation in a soup.

4. Medicinal Plants vs. Grocery Produce

Virtually, every plant contains some medicinal value. Sometimes the skin of the plant contains an entirely different medicinal value than the meat of the plant. In one embodiment, a soup combines powerful blends of vegetables, roots, mushrooms, legumes, herbs and spices to increase their therapeutic benefit and by combining then in a specific way, to create a potent synergy. By eating these transformative blends as soups, people boost their immune system, heal their gut, digestive issues (and various conditions related to that like irritable bowel syndrome) and protect themselves from serious conditions like cancer.

5. Chaunking vs. Sautéing

Chaunking opens up the medicinal properties, energies and flavors of certain spices and seals them at the same time. Seeds like cumin, mustard and coriander and chaunked together with other seasonings, like freshly ground ginger root, minced or whole chilies, in a heavy cast-iron skillet. Chaunks are added at the beginning of the soup.

6. Salt

Nutritionists often do not recommend soup in a diet because of the generally high sodium content of soup. In one embodiment, a soup described herein uses Himalayan pink salt. Himalayan pink salt in soups actually adds minerals that our bodies are missing, becoming a medicinal element. Himalayan pink salt contains 84 elements found in our bodies and by adding it to the soup blends, it actually becomes an essential missing element in regulation of blood pressure. It also regulates the water content in our bodies; promotes blood sugar health; promotes sinus health; prevents muscle health; promotes bone strength; regulates sleep patterns; and supports respiratory health.

### EXAMPLES

### Example 1

### Key Ingredient Combinations

The following represent embodiments of combinations of ingredients that produce a therapeutic benefit or benefits.
Butternut Squash and Red Lentils: produce a powerful Vitamin B6, folate and iron supplement.
Celery Root and Fennel and Leeks: satisfies daily requirement of Vitamin K, great source of Vitamin C, A.
Yam and Fennel with Cardamom and Fennel and Mustard Seeds: antioxidant and digestive aid, with glucosinolates, known to inhibit the cancer cells.
Sweet Potato and Coconut Oil/Milk and Holy Basil: satisfies daily requirement of Vitamin A, great source of Vitamin C.
Turmeric and Cumin and Shiitake Mushrooms and Lentils: powerful anti-cancer properties as well as at least 20 percent of daily requirement of folate and fiber in just one serving.
Kale and Sorrel: provide anti-cancer and cardiac properties as well as 206 percent of the recommended Vitamin A; 684 percent of the recommended Vitamin K; 134 percent of Vitamin C; and 26 percent of the Manganese.

### Example 2

### Veggie Healing Broth

This broth is full of cancer and disease-fighting properties. The soup is also a powerful anti-inflammatory, anti-bacterial supplement suitable, in one embodiment, for mitigating symptoms of a cold or flu or inhibiting the cold or flu. Each ingredient provides a therapeutic benefit, and combined together, provides a synergistic combination offering immune-boosting, cholesterol-lowering and healing.
½ bunch kale
½ bunch collards
½ bunch Swiss chard
½ bunch mustard greens
1 cup, cabbage
½ fennel bulb
½ medium onion
2 carrots, peeled and chopped
2 bok choy
3 inch piece daikon
5 whole dried shiitake mushrooms
3 garlic cloves, pounded
1 small knob ginger
1 small knob turmeric
½ fermented Asian pear with juice
cilantro
cilantro root
sweet potato skins
radish sprouts
Thyme
Parsley
Pour boiling water over washed and dried shiitake mushrooms and leave them to soak for at least 30 minutes. Strain the mushrooms through a fine-mesh strainer into a clean container.
Reserve water.
Combine the mushroom water with 2 quarts of spring water in a large pot and bring to soft boil on medium heat.
Grind ginger and turmeric and heat both in a dry heavy cast-iron skillet to release flavors and medicinal properties.
Add all ingredients except cilantro, thyme and parsley to the pot. If the veggies are too bulky, boil some more water and add to pot so all the veggies are covered. Simmer for 2 hours
Turn off heat under broth. Add cilantro, thyme and parsley. Steep 15 minutes. Strain into another large pot, pressing on solids in strainer.
Season with salt.
Add radish sprouts.
Cool 1 hour to allow flavors to mature.

### Example 3

### Sweet Potato Coconut Thai

The main ingredient here is sweet potato, packed with Vitamin A and beta-carotene, a powerful antioxidant that offers protection against cancer, asthma and heart disease. Combined with lemongrass, galangal, basil, coconut oil and milk, this soup is not just a great Vitamin A supplement and Beta-Carotene complex, this soup is anti-fungal, anti-bacterial that destroys free radicals and protects the body's cell repair.

Adult men generally need 3,000 IU (international units) of Vitamin A daily, per Linus Pauling Institute, while women need 2,333 IU Sweet potatoes provide 21,900 IU in one 4 oz. potato. Larger amounts of Vitamin A are beneficial for your health if taken in natural form but become toxic if taken as an over-the-counter supplement. This soup is also a great source of Vitamin C (¼ of daily recommended amount), Vitamin B (thiamin, riboflavin, niacin, pantothenic acid, B-6, choline and folate), Vitamin E (10 percent of daily recommended dose)
2 lbs. sweet potatoes, peeled and cut in big chunks
1 medium red onion, roughly chopped
2 cups coconut milk
1 tablespoon coconut oil
1 cup of Thai basil
1 knob of galangal
2 cloves of garlic
2 kaffir lime leaves, torn
1 teaspoon of vegan Thai red curry paste (more if you want the soup extra spicy)
1 lemongrass stalk, cut and pounded
Heat the oil in a heavy soup pot, add freshly ground galangal with minced garlic and onion and chaunk them to release flavor and healing properties.
Add the red curry paste, lemongrass, kaffir lime leaves and ½ cup of coconut milk. Stir and cook for 3 minutes.
Add sweet potatoes, mix them in and then add the remainder of coconut milk. Cook on medium heat for 20 minutes.
When the mixture starts bubbling, add boiled water, Thai basil, salt, reduce to simmer and cook until potatoes are very soft.
Remove from heat and let stand for 30 minutes to absorb flavors.
Remove kaffir leaves and lemongrass from the mixture and transfer the rest into the Vitamix.
Puree until smooth and silky.
Freshly squeezed lime juice is the perfect finish for serving.

### Example 4

### Lentil-Shitake

This soup has “anti-cancer” benefits as it combines a certain group of ingredients with scientifically proven anti-cancer properties. In addition, lentils protect the nervous system and improve mood, and are important for reducing stress that causes so many diseases. Shiitake mushrooms, turmeric, cumin, carrots and celery help to protect the immune system and slow (and even stop) cancer growth.

One serving of green lentils provides at least 20 percent of the recommended daily amount of folate and fiber, proven to act in several ways to lower cancer risk, including helping with weight control (excess body fat increases the risk of 8 cancers). Gut bacteria feed on fiber, which produces compounds that mat protect colon cells. Folate is essential for promoting healthy DNA and maintaining control of cell growth. Shiitake mushrooms' provide a tumor regression effect due to Lentinan, a beta-glucan polysaccharide, also known for its immune boosting properties.
1 cup small sprouted green lentils
2 medium carrots, cubed
2 celery stalks, cubed
4 spring scallions, cut diagonally into thin pieces
1 cup dried organic shiitake mushrooms, sliced
2 bay leaves
1 teaspoon turmeric powder
1 teaspoon cumin powder
1 tablespoon extra virgin olive oil
Himalayan pink salt
Wash the lentils and soak them overnight with warm water
Pour 5 cups of hot filtered water over washed and dried shiitake mushrooms and leave them to soak for at least 30 minutes. Strain the mushrooms through a fine-mesh strainer into a clean container. Reserve water.
Bring reserved mushroom water to boil. Add lentils and cook for about 20 minutes.
Meanwhile, heat the oil in a skillet over medium-low heat, add the scallions and sauté them for one minute until they turn bright green
Add the mushrooms and a little salt and sauté for another 2 minutes.
Add the turmeric and cumin and sauté for additional 2 minutes until scallions and mushrooms are coated with spices
While the lentils are cooking, add the mushroom-scallion mixture, plus carrots and celery into the pot, stir.
Cover and simmer for 15 minutes until lentils, mushrooms and veggies are al dente.
Taste and adjust salt to your taste.
Serve with a pinch of cayenne for kick and additional benefits.

### Example 5

### Yam+Fennel

This velvety, rich and delicately sweet soup is great for menopausal symptoms because of yam's naturally occurring properties that doctors+scientists believe act as natural alternatives to estrogen therapy. The soup has properties to inhibit hot flashes, osteoporosis, gallbladder problems, PMS and menstrual issues. Yams cooked with fennel, fennel seeds, cardamom and turmeric also create a powerful anti-diabetes, anti-cancer and cholesterol reducing food.
4 medium yams, scrubbed and halved
1 medium fennel bulb, cut in eighths
1 bunch of spring onions
1 tablespoon coconut oil
2 tablespoon cardamom powder
¼ teaspoon panch phoron
¼ teaspoon fennel seeds
¼ teaspoon mustard seeds
1 teaspoon turmeric
¼ teaspoon saffron
Himalayan Pink salt
Preheat oven to 400 degrees F., arrange yam halves on a baking sheet, sprinkle with 1 tablespoon of cardamom powder and roast until soft for about 20 minutes
Meantime, heat the coconut oil in soup pot, add mustard seeds, fennel seeds and panch phoron. When seeds begin to flutter, add spring onions, remainder of cardamom and turmeric. Mix.
Stir in the fennel, sauté the mixture until fennel is covered in spices.
Add the roasted yams, boiled water to cover and bring to simmer for about 30-45 minutes.
Allow soup to cool lightly to absorb flavors, then puree in the Vitamix until it's perfectly smooth.
Pour the soup into a clean pot, taste it and adjust seasonings to your taste.

### Example 6

### Celery Root-Fennel-Leek

This soup is prescribed for people suffering from digestive system disorders like ulcers, gastritis and indigestion. It improves liver and bladder functions and stimulates metabolism. The soup has high content of Vitamin B-6, A, K and C.

A single one-cup serving of leeks boasts 1,484 IU (international units) of vitamin A—64 percent of the daily intake for women and 49 percent for men. Getting enough vitamin A in the diet supports healthy blood cell development, including the growth of new red blood cells that transport oxygen, and white blood cells that fight infection. Vitamin A also benefits retinal health. Vitamin K benefits various tissue in the body, helping to regulate bloodflow, while low vitamin K levels can induce bleeding, which can negatively affect circulation. Getting enough vitamin K also activates osteocalcin, which is a protein essential for bone health. Leeks contain 42 micrograms of vitamin K per cup, celery root contains 45 mcg. This satisfies the daily vitamin K intake for women and almost entire amount of vitamin K for men. A 1-cup serving of fennel contains 10.4 milligrams of vitamin C, which is 17 percent of the daily values set by the FDA, and 838 International Units of vitamin A, which is also 17 percent of the FDA's recommended DV.
1 bunch spring onions, cut
2 garlic cloves, bruised
1 tablespoon coconut oil
2 leeks, cut
1 medium fennel bulb, quartered
1 medium celeriac root, peeled and cubed
Himalayan Pink Salt
Heat the coconut oil in a pot and sauté spring onions with garlic until greens turn bright green, approximately 2 minutes
Stir them with a pair of chopsticks dipped in HPS over a high heat
Add the leeks and sauté until tender for another 2-3 minutes
Add the fennel, sauté until tender for 3 minutes
Stir in celeriac, add more Himalayan Pink Salt, mix all the veggies in the pot and sauté for another few minutes.
Add 5 cups of boiling filtered water or enough to cover the veggies with about two inches of water above.
Soft boil on medium low for about 30 minutes, lower the heat and simmer another hour until all veggies are very soft.
Transfer everything to Vitamix and blend until smooth.
Serve hot.

### Example 7

### Butternut with Gingered Red Lentils

Butternut squash and red lentils supplement breast health. Vitamins and nutrients like Vitamins B6, C, A, carotenoids, folate and lots of iron also support bone and immune strength. Finally, the soup provides a significant source of protein and soothing for digestion.

The combination of butternut squash and red lentils provide an outstanding source of B vitamins, including vitamin B-6, thiamin and pantothenic acid. They are especially high in folate —also known as vitamin B-9—with 397 micrograms in just one cup. Adults should have 400 micrograms of folate each day, and eating 1 cup of this soup would fulfill this requirement. The body needs adequate folate to synthesize red blood cells and DNA and to aid in energy metabolism. A cup of this soup satisfies a daily requirement of iron, and by combining butternut with red lentils, allowing the body to absorb iron much easier. A cup of soup also supplies more Vitamin A than people need in a day
1 small Butternut squash (about 1½ lbs.), cut sideways
1 small onion, chopped
1 tablespoon coconut oil
½ teaspoon virgin olive oil
2 inches fresh ginger, sliced
1 medium heirloom tomato
1/2 cup sprouted red lentils
Himalayan Pink Salt
Wash and half squash lengthwise, scrape out the seeds and pulp.
Preheat over to 375 degrees F.
Cube the squash, season with salt and drizzle with olive oil.
Arrange on a baking sheet and roast for 25 minutes until flesh is fork-tender.
Reserve seeds and roast on the same baking sheet, without oil
In a heavy cast-iron pan chaunk ground ginger for 2 minutes
In a soup pot on medium heat, sauté the onion in coconut oil until translucent.
Add the ginger
Add the roasted squash, sauté for another 3 minutes.
Add the tomato, sprouted lentils and then boiled filtered water to cover
Cook on medium until soft boils, reduce the heat and simmer for 30 minutes to an hour until lentils are boiled over.
Let cool for another 30 minutes to absorb flavors
Transfer everything to Vitamix and blend until smooth.
Serve hot. Garnish with roasted seeds.

### Example 8

### Kale-Sorrel

This soup is a great cleansing soup that can lower cholesterol, support the immune system and is high in iron, Vitamins K and C. Kale together with sorrel provides 206 percent of the recommended Vitamin A, 684 percent of the recommended Vitamin K, 134 percent of Vitamin C and 26 percent of the Manganese. This soup can reduce the risk of heart disease and fights cancer. Studies show that combined with sorrel, it contains polyphenolic compounds, flavonoids and anthocyanins that seek out free radicals and zap them before they are able to mutate.
1 tablespoon coconut oil
1 bunch Tuscan kale, chopped
1 bunch (2 loosely packed cups) sorrel, chopped
1 medium potato
1 medium onion, cut in eighths
Himalayan Pink Salt to taste
In a soup pot on medium heat, sauté the onion in coconut oil until translucent.
Add the potato, salt and pepper and sauté for 3 minutes
Add the kale and stir; watch the greens turn bright green
Add 4-5 cups of boiled filtered water, bring to simmer and cook until potatoes are soft, about 30 minutes
Add sorrel and gently simmer for another 10 minutes
Carefully ladle soup into the Vitamix and blend until completely smooth.
Season with more salt if needed and serve immediately.

1. A supplement comprising a plurality of ingredients selected from one or more of vegetables, roots, herbs, spices, legumes in combination with a water broth, the plurality of ingredients selected for a nutritional benefit.

2. The supplement of claim 1, wherein the plurality of ingredients comprise kale, collards, Swiss chard, mustard greens, cabbage, fennel shitake mushrooms, turmeric, onion, carrots and the therapeutic benefit comprises at least one of immune system boosting and cholesterol lowering.

3. The supplement of claim 1, wherein the plurality of ingredients comprise sweet potatoes, coconut milk, coconut oil, lemongrass, galangal and basil and the therapeutic benefit comprises at least one of an anti-fungal benefit and an anti-bacterial benefit.

4. The supplement of claim 1, wherein the plurality of ingredients comprise lentils, shitake mushrooms, turmeric, cumin, carrots and celery and the therapeutic benefit comprises a protection of the immune system.

5. The supplement of claim 1, wherein the plurality of ingredients comprise yams, fennel, caradamon and turmeric and the therapeutic benefit comprises inhibition of menopausal symptoms.

6. The supplement of claim 1, wherein the plurality of ingredients comprise celery root, fennel, and leeks and the therapeutic benefit comprises inhibition of digestive system disorders.

7. The supplement of claim 1, wherein the plurality of ingredients comprise butternut squash and red lentils and the therapeutic benefit comprises one of breast health, bone strength and immune strength.

8. The supplement of claim 1, wherein the plurality of ingredients comprise kale and sorrel and the therapeutic benefit comprises one of cholesterol lowering and immune system support.

9. A method comprising:
combining a plurality of ingredients selected from one or more of vegetables, roots, herbs, spices, legumes with a water broth, the plurality of ingredients selected for a nutritional benefit; and
simmering the combination for a time period sufficient to release nutrients from the plurality of ingredients.

10. The method of claim 9, wherein the plurality of ingredients comprise kale, collards, Swiss chard, mustard greens, cabbage, fennel shitake mushrooms, turmeric, onion, carrots and the therapeutic benefit comprises at least one of immune system boosting and cholesterol lowering.

11. The method of claim 9, wherein the plurality of ingredients comprise kale, collards, Swiss chard, mustard greens, cabbage, fennel shitake mushrooms, turmeric, onion, carrots and the therapeutic benefit comprises at least one of immune system boosting and cholesterol lowering.

12. The method of claim 9, wherein the plurality of ingredients comprise sweet potatoes, coconut milk, coconut oil, lemongrass, galangal and basil and the therapeutic benefit comprises at least one of an anti-fungal benefit and an anti-bacterial benefit.

13. The method of claim 9, wherein the plurality of ingredients comprise lentils, shitake mushrooms, turmeric, cumin, carrots and celery and the therapeutic benefit comprises a protection of the immune system.

14. The method of claim 9, wherein the plurality of ingredients comprise yams, fennel, caradamon and turmeric and the therapeutic benefit comprises inhibition of menopausal symptoms.

15. The method of claim 9, wherein the plurality of ingredients comprise celery root, fennel, and leeks and the therapeutic benefit comprises inhibition of digestive system disorders.
